(19) United States
(12) Patent Application Publication
(10) Pub. No.: US 2024/0018865 A1
(43) Pub. Date: Jan. 18, 2024

(54) TBM ADAPTIVE SURROUNDING ROCK CLASSIFICATION METHOD ORIENTED TO ADVANCE RATE AND SAFETY

(71) Applicants: Shijiazhuang Tiedao University, Shijiazhuang (CN); Xinjiang Irtysh Investment and Development Co., Ltd, Urumqi (CN)

(72) Inventors: Lijie Du, Shijiazhuang (CN); Qingwei Li, Shijiazhuang (CN); Yalei Yang, Shijiazhuang (CN); Baoxin Liu, Shijiazhuang (CN); Quan Shi, Shijiazhuang (CN); Yongwei Quan, Shijiazhuang (CN); Xiangbo Zhao, Shijiazhuang (CN)

(73) Assignees: Shijiazhuang Tiedao University, Shijiazhuang (CN); Xinjiang Irtysh Investment and Development Co., Ltd, Urumqi (CN)

(21) Appl. No.: 18/083,501

(22) Filed: Dec. 17, 2022

(30) Foreign Application Priority Data
Jul. 18, 2022 (CN) 202210841946.5

(51) Int. Cl. E21B 47/047; E21B 49/00

(52) U.S. Cl. CPC E21B 47/047; E21B 49/006

(57) ABSTRACT

A TBM adaptive surrounding rock classification method includes following steps: establishing a TBM adaptability surrounding rock classification table; substituting the rock uniaxial compressive strength and a rock mass intactness index into the surrounding rock classification table to obtain basic classification of a surrounding rock; determining whether the grade of the surrounding rock needs to be corrected or not, and when correction is not needed, enabling the basic classification of the surrounding rock to be the final grade of the surrounding rock; when the correction is needed, performing rock burst correction or groundwater correction on the grade of the surrounding rock; determining which corrected classification of a rock burst correction result and a groundwater correction result is poorer in surrounding rock evaluation; and obtaining a conclusion, and taking the corrected classification with poorer surrounding rock evaluation as the final grade of the surrounding rock.

### CROSS-REFERENCE TO RELATED APPLICATION

This application claims priority to Chinese Patent Application No. 202210841946.5, filed on Jul. 18, 2022, which is hereby incorporated by reference in its entirety.

### TECHNICAL FIELD

The present disclosure belongs to the technical field of tunnel construction of full face rock tunnel boring machines (TBM for short), and in particular relates to a TBM adaptive surrounding rock classification method oriented to advance rate and safety.

### BACKGROUND

Tunnel surrounding rock classification methods with existing regulations and applications mainly face drilling and blasting method construction, classification is conducted according to surrounding rock stability, and there is no special surrounding rock classification standard for tunnel constructed through TBM method. However, surrounding rock conditions favorable to the drilling and blasting construction are not necessarily favorable to the TBM method. For example, for the surrounding rock conditions with extremely high compressive strength and excellent intactness, although the stability is excellent, the TBM is difficult in boring penetration, slow in advance rate, large in tool consumption, and poor in adaptability, such surrounding rock is poor surrounding rock for the TBM. Therefore, it is necessary to study and put forward a TBM construction adaptability surrounding rock classification method.

The TBM construction adaptability is that on the premise that the safety is guaranteed, the surrounding rock favorable to high advance rate is good surrounding rock, indicating that the TBM adaptability is good; on the contrary, the surrounding rock with the low advance rate is poor surrounding rock, indicating that the TBM adaptability is poor. Therefore, an advance rate-oriented TBM adaptive surrounding rock classification method is studied and provided.

Up to now, although there are some classification studies based on the boreability or penetration rate of surrounding rock, the methods are not yet mature, and more importantly, “boreability”, “penetration rate” and “advance rate” are not the same concepts. Boreability is a single index to express the difficulty of TBM penetration, but the advance rate for the surrounding rock which is easy to penetrate is not necessarily fast. For example, the surrounding rock with broken fault is easy to penetrate, but the advance rate is slow due to its easy collapse, high risk and large number of supports, therefore, the TBM boreability is good but TBM adaptability is poor. The penetration rate (mm/min or m/h) refers to the forward penetration rate of the TBM during boring and is an index when a cutter head of the TBM rotates and advances, while the TBM advance rate (m/day or m/week or m/month) is the footage advance rate of the TBM within a period of time, including both the boring time of the TBM and the shutdown time of the TBM, for example, the influence caused by footage delay due to the fact that the TBM stops for the support of broken surrounding rock is included.

### SUMMARY

A purpose of the present disclosure is to provide a TBM adaptive surrounding rock classification method oriented to advance rate and safety, which provides a practical and reliable method for analyzing the advance rate and construction period of the project feasibility study stage, the design stage and the construction bidding stage of the tunnel constructed by a TBM method.

In order to achieve the above purpose, it is provided a TBM adaptive surrounding rock classification method oriented to advance rate and safety, including following steps:
establishing a TBM adaptability surrounding rock classification table oriented to the advance rate and safety;
substituting rock uniaxial compressive strength and rock mass intactness index into the surrounding rock classification table to obtain surrounding rock basic classification;
determining whether a grade of surrounding rock needs to be corrected or not, and when correction is not needed, enabling the surrounding rock basic classification to be a final grade of the surrounding rock;
when the correction is needed, performing rock burst correction or groundwater correction on the grade of the surrounding rock;
determining which corrected classification of a rock burst correction result and a groundwater correction result is poorer in surrounding rock evaluation; and
obtaining a conclusion, and taking the corrected classification with poorer surrounding rock evaluation as the final grade of the surrounding rock.

Further, the advance rate in the surrounding rock classification table is obtained by collecting boring data of a large number of practical engineering cases on site and analyzing advance rate AR under different surrounding rock conditions through using a big data statistical analysis method, and the advance rate is divided into five classes: AR-I, AR-II, AR-III, AR-IV and AR-V, with division criteria as follows: the advance rate of greater than 20 m/d is classified as class AR-I, the advance rate of greater than 15 and smaller than or equal to 20 m/d is classified as class AR-II, the advance rate of greater than 10 and smaller than or equal to 15 m/d is classified as class AR-III, the advance rate of greater than 5 and smaller than or equal to 10 m/d is classified as class AR-IV, and the advance rate of less than or equal to 5 m/d is classified as class AR-V.

Further, TBM advance rate AR classes corresponding to various combinations of the uniaxial compressive strength UCS and the intactness index Kv of the surrounding rock are obtained by means of big data statistics and mathematical regression analysis, and the surrounding rock basic classification is obtained according to the compressive strength and the intactness index of the surrounding rock.

Further, the surrounding rock is basically divided into five grades which are excellent T-I, good T-II, medium T-III, poor T-IV, and extremely poor T-V.

Further, through the rock burst correction, degree of influence of different grades of rock burst on the advance rate and safety of the TBM is obtained by analyzing a relationship between rock burst grade of a rock burst tunnel section and the advance rate and safety, and the down-regulated grade of the surrounding rock of the rock burst tunnel section is determined on a basis of the surrounding rock classification table, thus a rock burst surrounding rock classification correction table is obtained.

Further, through the groundwater correction, by analyzing degree of influence of different types and different magnitudes of water inrush on the TBM advance rate and safety, a groundwater surrounding rock corrected classification table is obtained for the tunnel with groundwater on a basis of the surrounding rock classification table.

Due to the adoption of the structure above, compared with the prior art, the method provided by the present disclosure has the technical improvements that: based on the advance rate, not only can the difference of the difficulty of penetration caused by different compressive strength and intactness of the surrounding rock can be reflected, but also the difference of boring delay caused by supports due to different intactness or broken degree of the surrounding rock can be reflected, and moreover, the “good” and “poor” of the surrounding rock can be expressed fully, that is, TBM construction adaptability. Therefore, a TBM adaptive surrounding rock classification method is established oriented to the advance rate and based on big data analysis of the correlation between the advance rate and surrounding rock indices such as compressive strength and intactness of the surrounding rock, then the surrounding rock is basically classified. The correction is then carried out according to the rock burst or groundwater conditions of the surrounding rock; and after correction, the surrounding rock is evaluated to obtain the final grade of the surrounding rock. To sum up, a practical and reliable method is provided for analyzing the advance rate and construction period of the project feasibility study stage, the design stage and the construction bidding stage of the tunnel constructed through a TBM method.

### BRIEF DESCRIPTION OF THE DRAWINGS

The accompanying drawings are provided for further understanding of the present disclosure, these drawings are a part of the specification and used to explain the present disclosure together with the embodiments of the present disclosure, but not limit the present disclosure.

In the drawings:

FIG. 1 is a flow diagram in accordance with an embodiment of the present disclosure;

FIG. 2 is a surrounding rock classification table in accordance with an embodiment of the present disclosure;

FIG. 3 is a surrounding rock classification feature table in accordance with an embodiment of the present disclosure;

FIG. 4 is a rock burst surrounding rock classification correction table in accordance with an embodiment of the present disclosure;

FIG. 5 is a groundwater surrounding rock corrected classification table in accordance with an embodiment of the present disclosure;

FIG. 6 is a diagram illustrating relationship between TBM penetrations and rock uniaxial compressive strengths in different projects in accordance with an embodiment of the present disclosure;

FIG. 7 is a rectangular diagram illustrating influence of rock burst on advance rate in ABH project in accordance with an embodiment of the present disclosure.

### DETAILED DESCRIPTION

The exemplary preferred embodiments of the present disclosure are described below with reference to the accompanying drawings. It should be understood that the preferred embodiments described here serve only to illustrate and explain the present disclosure rather than limiting the present disclosure.

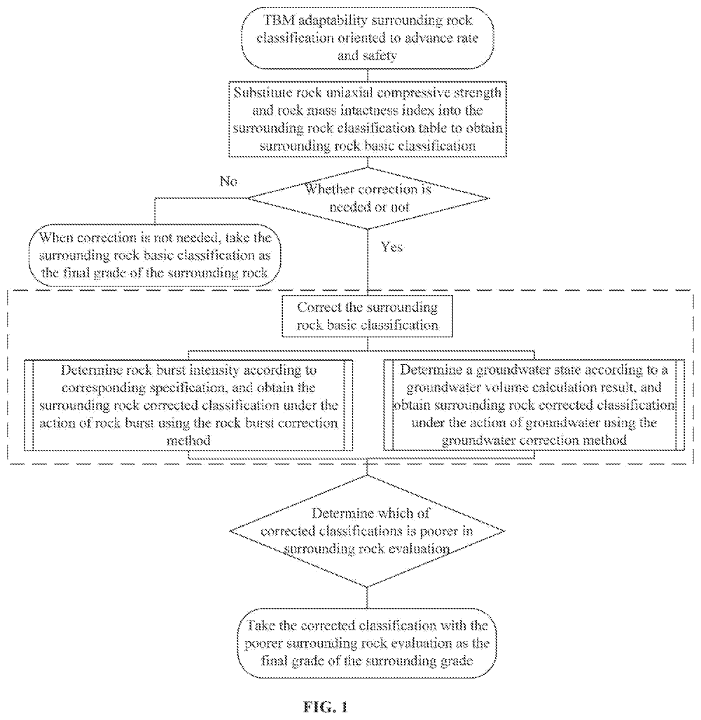

A TBM adaptive surrounding rock classification method under advance rate and safety, as shown in FIG. 1 to FIG. 5, includes the following steps:
S1, establishing a TBM adaptability surrounding rock classification table for the advance rate and safety;
S2, substituting rock uniaxial compressive strength and rock mass intactness index into the surrounding rock classification table in the step S1 to obtain surrounding rock basic classification;
S3, determining whether the grade of the surrounding rock needs to be corrected or not, and when correction is not needed, enabling the surrounding rock basic classification to be the final grade of the surrounding rock;
S4, when the correction is needed, performing rock burst correction or groundwater correction on the grade of the surrounding rock;
S5, determining which corrected classification of a rock burst correction result and a groundwater correction result is poorer in surrounding rock evaluation; and
S6, obtaining a conclusion, and taking the corrected classification with poorer surrounding rock evaluation as the final grade of the surrounding rock.

As a preferred embodiment of the present disclosure, the advance rate in the surrounding rock classification table is obtained by collecting boring data of a large number of practical engineering cases on site, and analyzing the advance rate AR under different surrounding rock conditions through using a big data statistical analysis method, and the advance rate is divided into five classes: AR-I, AR-II, AR-III, AR-IV and AR-V, with the division criteria as follows: the advance rate of greater than 20 m/d is classified as class AR-I, the advance rate of greater than 15 and smaller than or equal to 20 m/d is classified as class AR-II, the advance rate of greater than 10 and smaller than or equal to 15 m/d is classified as class AR-III, the advance rate of greater than 5 and smaller than or equal to 10 m/d is classified as class AR-IV, and the advance rate of less than or equal to 5 m/d is classified as class AR-V. The TBM advance rate AR classes corresponding to various combinations of the uniaxial compressive strength UCS and the intactness index Kv of the surrounding rock are obtained by means of big data statistics and mathematical regression analysis, and the surrounding rock basic classification is obtained according to the compressive strength and the intactness index of the surrounding rock. The surrounding rock is basically divided into five grades which are excellent T-I, good T-II, medium T-III, poor T-IV, and extremely poor T-V.

To further quantify the change rule of the advance rate along with the basic classification index of the surrounding rock, the advance rate for a part of bored sections of Dahuofang Project and EH Project is counted, and the mean value of the counted advance rates is listed in a Table 1. However, due to the limitation of the geology exposed by the construction, the advance rate distribution under the combination of the rock uniaxial compressive strength and rock mass intactness indices cannot be all counted. The surrounding rock basic classification cannot be completely obtained by only relying on the data in the Table 1, thus there is a need to supplement the advance rate distribution under the combination of the other rock uniaxial compressive strengths and rock mass intactness indices.

It is not difficult to find from the Table 1 that when the rock mass intactness is poor or better, the advance rate is high, while the rock mass intactness is relatively broken or broken, the advance rate decreases rapidly, indicating that the advance rate is more obviously changed along with the rock mass intactness. Therefore, the rock mass intactness is used as a control variable to discuss the advance rate under the combination of the different rock uniaxial compressive strengths and rock mass intactness indices.

TABLE 1
Mean Value of Advance Rate m/d at Partial
Sections of Dahuofang and EH Projects

| | UCS/MPa | | | |
|---|---|---|---|---|
| Intactness (Kv) | 15-30 | 30-60 | 60-90 | 90-120 |
| Intact (0.75-1) | — | — | 21.46 | 22.26 |
| Relatively intact (0.55-0.75) | — | 33.499 | 22.46 | 20.64 |
| Poor intact (0.35-0.55) | — | 36.759 | 13.21 | 14.8 |
| Relatively broken (0.15-0.35) | 5.972 | 9.04 | 8 | — |
| Broken (0-0.15) | — | 4.95 | — | — |

1. Rock Mass Intactness being Intact or Relatively Intact

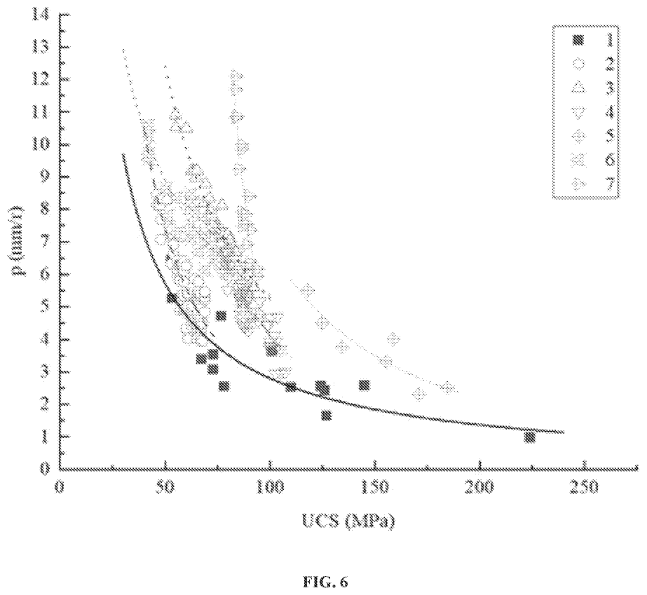

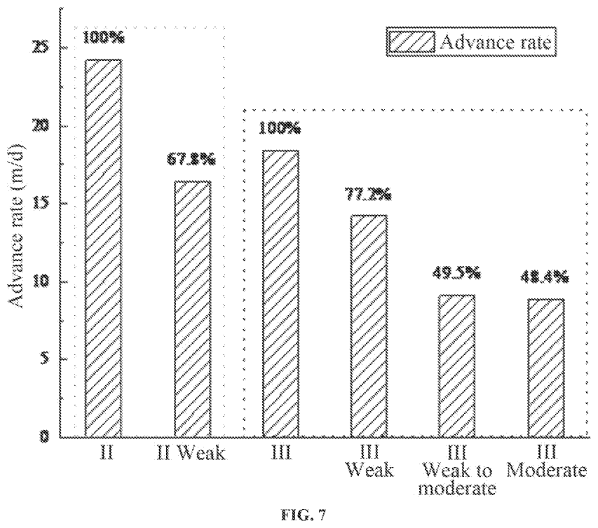

When the rock mass intactness is relatively intact to intact (the intactness index Kv is between 0.55-1), the TBM can obtain a higher utilization rate due to less time consumption in supporting, and therefore the influence of the penetration rate on the advance rate is larger. The penetration refers to a depth of a hob cutting into the rock mass after a TBM cutter head rotates for one circle, a rotational speed refers to a rotational speed of the TBM cutter head, and the penetration rate can be calculated by multiplying the penetration by the rotational speed of the cutter head. The rotational speed of the cutter head is a manually set parameter and is generally slight in change, and the penetration is a parameter generated in the construction process and is highly sensitive to geology, so the study on the penetration rate can be started from the penetration. In FIG. 6, the change of the TBM penetration with the rock uniaxial compressive strengths in seven projects (the data points shown in FIG. 6 are all chosen from the rock mass intactness index range of 0.55-1) is provided, it can be found that the penetration is inversely related to the rock uniaxial compressive strength and the curve type is similar to the power function, and the results of fitting with the least square method are shown in Table 2.

TABLE 2
Penetration Fitting Formula of Different Engineering

| Serial | | TBM | | Penetration fitting | |
|---|---|---|---|---|---|
| number | Data source | diameter/m | TBM type | formula | R² |
| 1 | Zhuxi Reservoir Project | 4.03 | Open | y = 309.1 · x\hat{ }(−1.024) | 0.792 |
| 2 | Dahuofang Project | 8.03 | Open | y = 3048.6 · x\hat{ }(−1.541) | 0.737 |
| 3 | Songhuajiang River Diversion | 8.5 | Open | y = 1975.85 · x\hat{ }(−1.292) | 0.931 |
| | Project in Jilin | | | | |
| 4 | Huanren-Jian Tunnel | 8.5 | Open | y = 44230 · x\hat{ }(−2.02) | 0.733 |
| 5 | Nabang Hydropower Station | 4.53 | Open | y = 15355 · x\hat{ }(−1.675) | 0.827 |
| 6 | Gaoligongshan Tunnel | 9.03 | Open | y = 200.48 · x\hat{ }(−0.794) | 0.778 |
| 7 | Shenzhen Metro Line 6 | 6.2 | Double-shield | y = (3 × 10¹²) · x\hat{ }(−5.921) | 0.863 |

In FIG. 6, penetration fitted curves of the TBM are not collinear due to the fact that the rock mass intactness index and TBM single-tool thrust are different in different projects. To reduce the influence of the rock mass intactness index and the thrust difference on the penetration as much as possible, seven fitted curves need to be averaged. An approximate penetration prediction formula (formula 1-1) is obtained after average calculation. The mean penetration for different rock uniaxial compressive strength ranges can be calculated by utilizing a formula (1-2), and corresponding calculation results are listed in Table 3. The penetration rate can be calculated by using a formula (1-3).

$$p = 6127.7 \times U C {S}^{- 1.569} \qquad ( 1 - 1 )$$

$$\overline{p} = \frac{{\int }_{a}^{b} 6127.7 \times U C {S}^{- 1.569} {d}_{( U C S )}}{b - a} \qquad ( 1 - 2 )$$

$$PR = p \times n \qquad ( 1 - 3 )$$

where:
PR denotes penetration rate, mm/min;
p denotes penetration, mm/r;
n denotes rotational speed, rpm;
UCS denotes rock uniaxial compressive strength, MPa;
a and b respectively denote an upper limit and a lower limit of integral interval.

TABLE 3
Mean Value of Penetration in Intact to Relatively Intact Rock Mass

| UCS Interval/MPa | Mean value of penetration/(mm · r⁻¹) |
|---|---|
| 30-60 | 16.89 |
| 60-90 | 7.20 |
| 90-120 | 4.19 |
| 120-150 | 2.81 |
| 150-180 | 2.04 |
| 180-210 | 1.57 |
| 210-240 | 1.25 |
| 240-270 | 1.03 |
| 270-300 | 0.86 |
| — | — |

After counting the projects listed in the Table 2, it is found that on the premise that the rock mass intactness is intact or relatively intact, the rotational speed of the TBM gradually increases within the rock uniaxial compressive strength range of 30 to 210 MPa, the boring utilization rate of the TBM is firstly increased and then decreased within the rock uniaxial compressive strength range of 30 to 240 MPa, and the rotational speed and the utilization rate of the TBM in different rock uniaxial compressive strength intervals are shown in the Table 4. The continuous increase of the penetration rate is due to the large penetration that can be obtained when the uniaxial compressive strength of the rock is low. To control the amount of slag entering the cutter head to be within the bearable range of a belt conveyor, the rotational speed of the cutter head needs to be actively decreased. It is hard to obtain large penetration when the compressive strength of the rock is high, and the rotational speed needs to be increased in order to increase penetration rate as much as possible. As the related data of the rotational speed within the range of 210 to 300 MPa is not collected, the mean rotational speed within the range of 180 to 210 MPa is used for replacing the rotational speed within the range of 210 to 300 MPa in a unified mode. The increase of the utilization rate is that, on one hand, the influence that the rock mass intactness and the rock uniaxial compressive strength in the statistical projects have a direct proportion relation is not completely eliminated; the increase of the utilization rate is that, on the other hand, the self-stability of the surrounding rock is enhanced with the increase of the rock uniaxial compressive strength, and the downtime caused by supporting is shortened. The reason of utilization decreasing after increasing is that the increase in the rock uniaxial compressive strength causes increased tool consumption and TBM system failures, which increases the time spent on tool replacement and equipment maintenance. As the related data of the utilization rate within the range of 240 to 300 MPa is not collected, the mean utilization rate within the range of 210 to 240 MPa is used for replacing the utilization rate within the range of 240 to 300 MPa in a unified mode.

The advance rate can be obtained after the penetrations, the rotational speeds and the mean utilization rates for different rock uniaxial compressive strength ranges are obtained, and corresponding results are listed in Table 4. As can be known from the calculation results, the advance rate continues to decrease as the uniaxial compressive strength of rock increases, and the surrounding rock grade in the new corresponding surrounding rock classification method is getting lower, that is, the rock mass quality is getting more unfavorable for TBM boring under the new evaluation system.

TABLE 4
Calculation Table of Advance Rate in Intact to Relatively Intact Rock Mass

| | Mean value | | | | | |
|---|---|---|---|---|---|---|
| | of | Rotational | | Advance | Class of | Grade of |
| UCS | Penetration/ | speed/ | Utilization | rate/ | advance | surrounding |
| interval/MPa | (mm · r⁻¹) | (r · min⁻¹) | rate | (m · d⁻¹) | rate | rock |
| 30-60 | 16.89 | 6.23 | 42.7% | 64.71 | AR-I | T-I |
| 60-90 | 7.20 | 6.36 | 49.1% | 32.37 | AR-I | T-I |
| 90-120 | 4.19 | 6.49 | 50.7% | 19.85 | AR-II | T-II |
| 120-150 | 2.81 | 6.63 | 48.5% | 13.00 | AR-III | T-III |
| 150-180 | 2.04 | 6.77 | 43.5% | 8.67 | AR-IV | T-IV |
| 180-210 | 1.57 | 6.86 | 42.1% | 6.53 | AR-IV | T-IV |
| 210-240 | 1.25 | 6.86 | 39.0% | 4.83 | AR-V | T-V |
| 240-270 | 1.03 | 6.86 | 39.0% | 3.96 | AR-V | T-V |
| 270-300 | 0.86 | 6.86 | 39.0% | 3.33 | AR-V | T-V |

In conjunction with the Dahuofang Project and the EH Project, the calculation results in Table 4 have the following problems.

(1) The measured penetration when the rock uniaxial compressive strength ranges from 30-90 MPa is 5.88 mm/r, while the predicted penetration is larger than the measured value and deviates significantly. This is because excessive penetration may generate a large amount of rock slag and there is an upper limit in the slag feeding amount of the cutter head, a TBM operator usually reduces the penetration to control the penetration rate in order to avoid extra abrasion between a tool and the rock slag. However, when the rock uniaxial compressive strength is 30 to 90 MPa, the mean advance rate exceeds 20 m/d, and the surrounding rock belongs to T-I surrounding rock.

(2) When the rock uniaxial compressive strength is within 90 to 120 MPa, the mean value of actual advance rate is 21.45 m/d, and the calculated advance rate is 7.5% smaller than the actual advance rate. As the error is small, the calculated advance rate can be deemed to be reliable. However, in the classification, the surrounding rock in such range should be evaluated as the T-I surrounding rock according to the actual advance rate.

(3) The rock uniaxial compressive strengths in the selected construction sections of the Dahuofang Project and the EH Project are basically concentrated within 30 to 120 MPa, but in combination with the application conditions of the TBM in the high-strength rock sections of the water-conveying tunnels of the Taizhou City Zhuxi Reservoir Project and the Nabang Hydropower Station, when boring in the surrounding rock with the uniaxial compressive strength of 200 MPa or above, the mean value of penetration is 0.98 mm/r, and the mean value of the advance rate is 3.5 m/d. Therefore, it can be considered that the predicted penetration and calculated advance rate in Table 4 and 5 are basically in line with the reality.

In addition, the calculated advance rate can be obtained on the premise that the rock mass intactness index is 0.55 to 1, while the intactness index of 0.55 to 1 in the new surrounding rock classification is divided into two classes. As higher penetration can be obtained in the surrounding rock with low rock mass intactness, when the rock uniaxial compressive strength is more than 120 MPa, the advance rate in the relatively intact surrounding rock should be higher than that in the intact surrounding rock by one class.

2. Rock Mass Intactness being Poor Intact

When the rock mass intactness index is within the range of 0.35 to 0.55, that is the rock mass intactness is poor intact, there are two supporting forms in the TBM construction: one is to adopt “anchor bolt+reinforcing mesh+sprayed concrete” for supporting, and the other is to adopt “anchor bolt+steel arch+reinforcing mesh+sprayed concrete” for supporting.

In the Dahuofang Project, the intactness indices of the surrounding rocks with poor rock mass intactness are mostly within the range of 0.45 to 0.55, the surrounding rock has self-stability during construction, but there are occasional falling blocks and cavities in the vault, so the “anchor bolt+reinforcing mesh+sprayed concrete” is used for supporting. In the EH project, the surrounding rock with the intactness index of 0.45 to 0.55 also employs the “anchor bolt+reinforcing mesh+sprayed concrete” for supporting, but when the intactness index is smaller than the surrounding rock is supported by using the “anchor bolt+steel arch+steel bar row+sprayed concrete”. This is because that the number and scope of fallen blocks and collapsed cavities in the vault increases with the decrease of the intactness index, and the anchor pullout resistance alone cannot provide sufficient supporting force, and the debris and easy-to-fall dangerous rocks may pose a greater threat to the safety of construction personnel and equipment if effective support is not formed quickly after the shield is released.

As the intactness index of more than 70% of data samples of the Dahuofang Project in the poor rock mass intactness range is greater than 0.5, the advance rate of the Dahuofang Project is obviously higher than that of the EH Project, but when the rock mass intactness index is 0.45, the mean TBM advance rate of the Dahuofang Project is 15.07 m/d. In the samples selected from the EH Project, the advance rate in the section with the intactness index of 0.35 to 0.45 is in the range of to 15 m/d, and the advance rate in the section with the intactness index of 0.45 to 0.55 is in the range of 15 to 20 m/d. As the data distribution of the EH Project is more uniform and stronger in representation, the EH Project is used as the sample for classifying the surrounding rock.

After taking the influence of the rock uniaxial compressive strength on TBM construction into consideration, when the rock mass intactness index is 0.45 to 0.55, the surrounding rock with the rock uniaxial compressive strength of 30 to 150 MPa is grade T-II, and the surrounding rock with the rock uniaxial compressive strength of more than 150 MPa is grade T-III. When the rock mass intactness index is 0.35 to 0.45, the surrounding rock is uniformly grade T-III.

3. Rock Mass Intactness being Broken or Relatively Broken

The TBM can reach a higher penetration with the decrease of the rock mass intactness, and at this moment, the most major limiting factor of the TBM advance rate is no longer the penetration rate but the boring utilization rate.

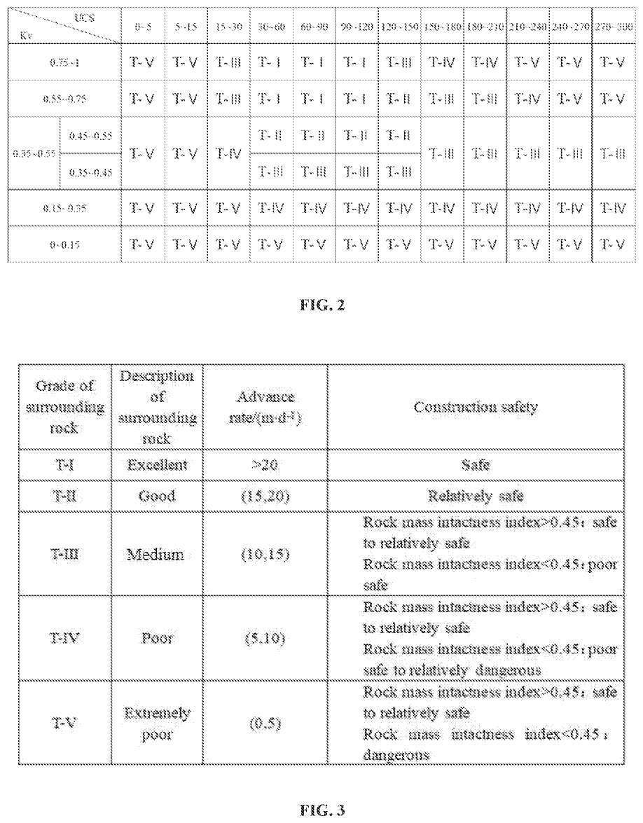

When the rock mass intactness is relatively broken to broken, the “anchor bolt+steel arch+reinforcing mesh+sprayed concrete” supporting is generally selected as the supporting mode for the surrounding rock. To guarantee the mounting precision, when the steel arch is used for supporting, the TBM is usually stopped and waits for the accumulated slag in the inverted arch to be cleaned. Furthermore, as the usage frequency of anchor drill rigs and steel arch installers increases, the number of failures may also increase, resulting in the prolonging of the downtime caused by mechanical failure. As the side wall of the tunnel is broken, a phenomenon that a gripper slips occurs in the EH Project. Therefore, the TBM advance rate is mainly limited by the boring utilization rate when penetrating through relatively broken and broken rock masses. As can be known from the conditions comprehensively reflected in FIG. 1 to FIG. 2, no matter which range the rock uniaxial compressive strength is in, the advance rate is mainly distributed in the range of 5 to 10 m/d when the rock mass intactness is relatively broken (the intactness index Kv is within the range of 0.15 to 0.35), and the rock mass belongs to the T-IV surrounding rock. The advance rate is mainly distributed in the range of 0 to 5 m/d when the rock mass intactness is broken (the intactness index Kv is between 0 and 0.15), and the rock mass belongs to T-V surrounding rock.

The influence of high ground stress on the TBM construction can be divided into two issues, one is large deformation and the second is rock burst. The TBM jamming phenomenon occurs for many times when crossing phyllite in Gaoligongshan Tunnel, this is because the uniaxial compressive strength of the phyllite encountered in the construction is about 15 MPa, large deformation phenomenon can occur even if the surrounding rock is relatively intact. The TBM3 frequently encounters the problem of machine jamming in the KS section of Xinjiang YE Project, this is because the tunnel crosses a fault fracture zone for multiple times and the broken rock mass is largely deformed by the ground stress. Therefore, the ground stress is not the only cause of large deformation. Large deformation in the TBM construction is also affected by the rock uniaxial compressive strength and the rock mass intactness, which can be induced by low rock strength or low rock mass intactness. In the surrounding rock basic classification above, the surrounding rock with the rock uniaxial compressive strength smaller than 15 MPa and the rock mass intactness being broken (the rock mass intactness index is smaller than 0.15) has been divided into grade T-V with the worst rock mass quality, and the grade of the surrounding rock cannot be down-regulated even if the large deformation problem occurs again. However, the rock burst problem may cause the change in the grade of the surrounding rock, so it is necessary to explore the influence of rock burst on TBM construction.

Rock burst is a violent energy release phenomenon and usually occurs in the surrounding rock with high ground stress occurrence and hard and intact rock, and its formation mechanism has not been completely ascertained, but a series of explanation theories such as a strength theory, an energy theory and a rigidity theory have been developed. A large amount of strain energy is accumulated in the rock mass under the action of ground stress, a free face is formed with the tunnel excavation, thus the energy in the rock mass is rapidly released, and the generation of craters on the wall of the tunnel is accompanied with the phenomena such as rock ejection, rock powder injection, shock wave. The rock burst can be divided into weak rock burst, moderate rock burst, intense rock burst and extremely intense rock burst according to the caused damage severity. The expression of different intensities of rock burst is listed in the Table 5.

TABLE 5
Expression of Different-Intensity Rock Burst of Underground Engineering

| | | | Intense rock | Extremely intense |
|---|---|---|---|---|
| Source | Weak rock burst | Moderate rock burst | burst | rock burst |
| Solution of | Tunnel wall is | Tunnel wall is burst | Tunnel wall is | Tunnel wall is burst |
| Tianshengqiao | partially split. | in patches, and most | burst in large | in large areas |
| Rock Burst | | rock fragments are | areas, and rock | continuously, and |
| Research | | separated from the | fragments are | ejection of massive |
| Group | | matrix. | separated from | rock fragments |
| | | | the matrix, | occurs. |
| | | | and ejection or | |
| | | | falling occurs. | |
| Solution of | Burst loosing, | The phenomenon of | Violet burst | Violet burst |
| Erlangshan | peeling off | burst loosing and | ejection occurs. | ejection, even |
| Ground Stress | | peeling off is | | throwing. |
| Research | | serious, and a little | | |
| Group | | of ejection occurs. | | |
| Solution of | Burst loosing, | The phenomenon of | Violet burst | Violet burst |
| Diversion | peeling off | burst loosing and | ejection occurs. | ejection, even |
| Tunnel at | | peeling off is | | throwing. |
| Jinping II | | serious, and a little | | |
| Hydropower | | of ejection occurs. | | |
| Station | | | | |
| Research | | | | |
| Group | | | | |
| Code for | Burst and fall | The phenomenon of | Large areas of | Most of |
| Engineering | phenomenon | rock burst and fall is | surrounding | surrounding rocks |
| Geological | occurs in the | obvious, and the | rock are burst, | at cavern section |
| Investigation | surface area of | damage range is | strong ejection | are severely burst, |
| of Water | surrounding rock. | large, and the | occurs, rock | severe ejection of |
| Resources and | Rock burst has an | influence depth is | mass ejection | massive rock |
| Hydropower | influence depth of | 0.3 to 1 m. | and rock | fragments occurs, |
| (GB50487- | 0.1 to 0.3 m. | | powder ejection | and the influence |
| 2008) | | | occur, and the | depth is greater |
| | | | influence depth | than 3 m. |
| | | | is 1 to 3 m. | |

4. Rock Burst Intensity Criterion

Ground stress is driving force generated by rock burst, which determines the direction and energy of the rock burst. Rock strength is an internal cause of rock burst, and the higher the rock strength is, the larger the elastic potential energy capable of being stored in the rock is. The rock burst intensity is jointly affected by the ground stress and the rock strength, and thus the ground stress and the rock strength are feasible to serve as the criteria of the rock burst intensity.

Zhenyu Tao counted the rock burst phenomena encountered in the hydropower engineering, and proposed, based on related research, to take a ratio of the rock uniaxial compressive strength to the maximum principal stress of ground stress as a rock burst criterion. Linsheng Xu, etc. summarized the rock burst occurrence rule in the Erlangshan Tunnel and found that there is a relationship between a ratio of the maximum tangential stress of the tunnel wall to the rock strength and the rock burst intensity. E.Hoke also took this ratio as the rock burst criterion. Code [20] and Code [21] also give the criteria for rock burst intensity. Code [20] takes a ratio of the rock uniaxial compressive strength to the initial stress perpendicular to the axis of the tunnel as a criterion, and Code [21] takes a ratio of the rock uniaxial compressive strength to the maximum principal stress as a criterion.

By summarizing the different methods above in a Table 6, it can be found that there is a big difference between the different methods, and thus the rock burst intensity should be determined with reference to the applicable code of the project at the investigation stage.

TABLE 6
Comparison of Rock Burst Criteria of Different Methods

| | Tao Zhengyu | Xu Linsheng | E. Hoke | | |
|---|---|---|---|---|---|
| Rock burst intensity | approach | approach | approach | Code[20] | Code[21] |
| Weak rock burst | 5.5~14.5 | 2~3.3 | 2.4~2.9 | 4-7: | 4-7 |
| Moderate rock burst | 2.5~5.5 | 1.4~2 | 1.8~2.4 | Occasional | 2-4 |
| | | | | rock burst; | |
| Intense rock burst | <2.5 | <1.4 | 1.4~1.8 | Smaller than | 1-2 |
| Extremely intense | | | <1.4 | 4: frequent | <1 |
| rock burst | | | | rock burst | |

Note: the criteria in the table are unified as the ratio of the ground stress to the rock uniaxial compressive strength. Depending on the method, the ground stress may refer to the maximum principal stress, the maximum tangential stress of the tunnel wall, or the maximum initial stress perpendicular to the tunnel axis.

5. Analysis of Influence of Rock Burst on TBM Advance Rate

An open type TBM having a diameter of 8.02 m was adopted for construction in the Hanjiang-to-Weihe River Water Diversion Project, during the boring of pile numbers K28+085 to K40+434, 795 rock bursts accumulatively occurred in total, and the rock uniaxial compressive strength at the sections with frequent rock burst was 100 to 200 MPa. Different intensities of rock burst have different influences on TBM advance rate. Table 7 shows the influence of the rock burst on the advance rate in different grades of surrounding rocks under the HC surrounding rock classification method. An open type TBM having a diameter of 6.53 m was adopted for construction in the ABH Project, during the boring of pile numbers K11+569 to K14+203, rock bursts occurred multiple times. Although the buried depth of the ABH Project is greater than that of the Hanjiang-to-Weihe River Water Diversion Project, the intensity of the rock burst is lower than that of the Hanjiang-to-Weihe River Water Diversion Project, this may be that the latter is more affected by the structural stress. FIG. 7 illustrates the influence of rock burst on the advance rate in different grades of surrounding rocks under the HC surrounding rock classification method in the ABH Project.

TABLE 7
Influence of Rock Burst on Advance Rate in Hanjiang-
to-Weihe River Water Diversion Project m/d

| Type of | | | | | | |
|---|---|---|---|---|---|---|
| surrounding | No | Weak | Weak to | Moderate | Moderate | Intense |
| rock (HC | rock | rock | moderate | rock | to intense | rock |
| method) | burst | burst | rock burst | burst | rock burst | burst |
| I | 7.0 | 6.8 | 6.2 | 5.3 | 3.9 | 3.2 |
| II | 12.9 | 9.9 | 9.5 | 5.8 | | |
| III | 7.6 | 6.0 | 6.3 | 3.9 | | |

From the instances of the Hanjiang-to-Weihe River Water Diversion Project and the ABH Project, it is not difficult to discover that the advance rate gradually decreases as the intensity of the rock burst increases, mainly due to safety considerations. When the rock burst occurs on the tunnel face and the shield, the ejected rocks caused by rock burst are prevented by the cutter head and the shield, and the safety of the construction workers can be guaranteed. However, the stability of the surrounding rock may be reduced due to a rock loose circle caused by rock burst, and supporting needs to be reinforced to reduce the possibility of hurting people by falling rocks. In addition, the rock burst position is not only limited in the protection range of a TBM shield, the delayed rock burst may occur in an L1 area, an L2 area and even the position where a back-up gantry is located, and the supporting also needs to be reinforced to prevent casualties and equipment damage. Rock burst mostly occurs in the hard and intact surrounding rocks, the supporting strength in such surrounding rocks is usually low, while the supporting strength has to be increased to cross a rock burst section safely, but the increase of the supporting strength causes the increase of the time spent on supporting, which is the main factor that the advance rate of the rock burst section is slower than that of a rock burst-free section. In addition to the increase of time spent on supporting, increased tool damage, mechanical equipment damage, dangerous rock disposal, slag removal, and reinforcement at the supporting position of the gripper also contribute to lower TBM utilization rate.

6. Surrounding Rock Grade Correction Under Rock Burst

Weak rock burst has a limited influence on TBM construction. From the aspect of supporting, the section with weak rock burst is generally supported using “anchor bolt+reinforcing mesh+sprayed concrete”, such supporting mode is simple in construction and less in time consumption. From the aspect of safety, the weak rock burst does not generate rock ejection phenomenon, and the depth of the crater is usually smaller than 0.5 m, such that the safe crossing can be achieved by means of the shield and the support, and the influence on personnel and equipment is small. By combining the Table 7 and FIG. 7, it can be found that the construction adaptability rating of the surrounding rock with the construction adaptability rating of T-I, T-II and T-III in the rock burst-free section is reduced by one grade after being affected by the weak rock burst, and the advance rate of the surrounding rock with the construction adaptability rating of T-IV in the rock burst-free section is reduced but is free of degradation after being affected by the weak rock burst.

Moderate rock burst has a deeper influence on the TBM construction than the weak rock burst. From the aspect of supporting, the moderate rock burst section is generally supported using “anchor bolt+steel bar row+steel arch+sprayed concrete”, such supporting mode is consistent with the type of supporting selected in the broken surrounding rock, which is large in construction difficulty, and time-consuming. The moderate rock burst is accompanied by a small amount of rock ejection phenomenon, craters having a depth of 0.5 to 1 m may be formed in the tunnel wall, and large-area rock loosening may occur. In consideration of safety, the threat of the moderate rock burst to personnel and equipment in the TBM construction is still within a controllable range, and safe crossing can be achieved by reasonably selecting a supporting mode. By combining the Table 7 and FIG. 7, it can be found that the construction adaptability rating of the surrounding rock with the construction adaptability rating of T-II in the rock burst-free section is reduced by two grades after being affected by the moderate rock burst, and the construction adaptability rating of the surrounding rock with the construction adaptability rating of T-III in the rock burst-free section is reduced by one grade after being affected by the moderate rock burst, while after being affected by the moderate rock burst, some of surrounding rocks with the construction adaptability rating of T-IV in the rock burst-free section are reduced and some are not. There is no moderate rock burst in the surrounding rock with the construction adaptability rating of T-I, but with reference to the change rule of weak rock burst, the influence on the surrounding rock with the construction adaptability rating of T-I should be similar to that on the surrounding rock with the construction adaptability rating of T-II, and the construction adaptability rating is reduced by two grades. The surrounding rock with the construction adaptability rating of T-IV, after being affected by the moderate rock burst, is inconsistent in degradation, but after averaging the advance rates under the moderate rock burst, it can be discovered that the mean advance rate is reduced by one grade compared with that in the case of no rock burst, and the construction adaptability rating should be reduced by one grade in a relatively conservative manner due to the consideration of the safety.

Intense rock burst and extremely intense rock burst have a significant influence on the TBM construction, and inadvertent handling will likely lead to project failure. Due to the large release of energy from the rock burst, the conventional means of support cannot fully satisfy the protection requirements, and a special treatment is required. Rock burst energy can be reduced in advance by applying advanced stress release holes, irrigating and blasting. Part of energy generated when rock burst occurs can be absorbed by means of flexible supports such as the energy-dissipating anchor bolts and energy-dissipating steel arches, and more deformation time can be provided for the surrounding rock by controlling the daily footage, thereby making the rock burst occur in the protection range of the shield as much as possible; furthermore, the shield is subjected to strength reinforcement treatment. Safe crossing is a primary consideration factor in the construction of the sections with intense rock burst and extremely intense rock burst, and the advance rate should give place to safety. Therefore, the grade of the surrounding rock in the sections with the intense rock burst and the extremely intense rock burst should be rated as the lowest grade, i.e., the T-V surrounding rock.

The influence of the rock burst is introduced into the surrounding rock basic classification, and the grade after correction is shown in FIG. 2. It should be noted in particular that in the Hanjiang-to-Weihe River Water Diversion Project, the ABH Project and the KS Section of the EH Project, the rock burst phenomenon usually occurs in the surrounding rock with rock uniaxial compressive strength greater than 60 MPa and rock mass intactness index greater than 0.45.

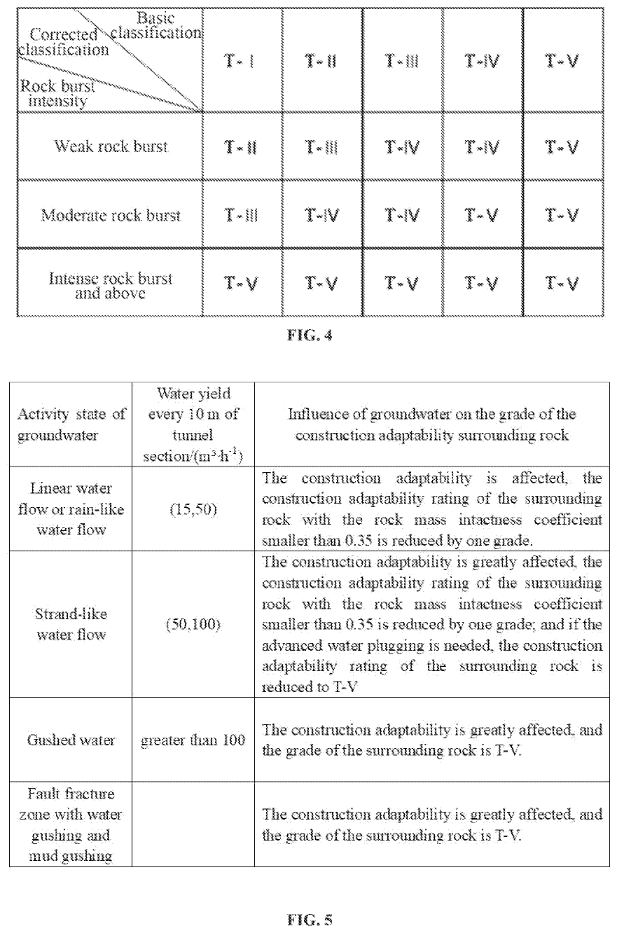

Through the rock burst correction, the degree of influence of different grades of rock burst on the advance rate of the TBM is obtained by analyzing a relationship between the rock burst grade and the advance rate of the rock burst tunnel section, and the down-regulated grade of the surrounding rock of the rock burst tunnel section is determined on the basis of the surrounding rock classification table, thus a rock burst surrounding rock classification correction table as shown in FIG. 4 is obtained.

Groundwater is an adverse influencing factor in tunnel engineering, and its activity state has been discussed by many classification methods. In Standard for Engineering Classification of Rock Mass (GBT 50218-2014), the activity states of groundwater are divided into a wet or dripping state, a rain-like or linear water discharging state, and a water gushing state, and the water yields of every 10 meters of tunnel length in the three states are respectively as follows: smaller than or equal to 25 L/min, larger than 25 L/min and smaller than or equal to 125 L/min, and larger than 125 L/min. In Code for Hydropower Engineering Geological Investigation of Water Resources and Hydropower (GB50287-2008), the water discharging states are divided into three types: water seepage to dripping, linear water flow and gushed water, and the division criteria of the water yield of each state is the same as that of the Standard for Engineering Classification of Rock Mass (GB/T 50218-2014). The drilling and blasting method is poor in adaptability to groundwater, the working environment in the tunnel is further deteriorated when the groundwater exists, and efficiency of procedures such as drilling, detonating and slag removing is reduced due to the influence of the groundwater. The TBM is subjected to waterproof design and provided with a drainage system, such that the TBM can continue to work in a case of less groundwater. There is a need to re-establish the classification of groundwater activity state for the characteristics of the TBM. In combination with the actual application of the TBM in Lingnan Section Water Transfer Tunnel of the Hanjiang-to-Weihe River Water Diversion Project, the Gaoligongshan Tunnel, the Taizhou City Zhuxi Reservoir Water Diversion Tunnel, the KS section of the EH Project and the SS section of the EH Project, the activity states of groundwater in construction can be divided into a dry state, a wet to water-seepage state, a dripping state, a linear water flow state or rain-like water flow state, a strand-like water flow state, and a water gushing state. The front four activity states of the groundwater have limited influence on the construction, belonging to general activity states of the groundwater. The influence of the water gushing state is large and serious, so it is called hazard-type activity state of the groundwater.

7. Influence of General Activity State of Groundwater on TBM Construction

The groundwater is not developed when the activity state of groundwater is dry, and the structural plane of the rock mass is not subject to the lubricating effect of water. In literatures, the boring in the dry tunnel section may cause forced increase of water spraying amount of the cutter head, temperature rise of the cutter head, accelerated abrasion of the cutter head, increased dust content in the tunnel and increased failure rate of a ventilation and dust removal system, leading to adverse effects on TBM construction. However, compared with the safety positive benefits brought by the dry tunnel section, the adverse effects brought by the tunnel section are negligible, and therefore the grade of the surrounding rock is not reduced when the activity state of groundwater is dry.

The water yield of every 10 meters of the tunnel section is below 5 m³/h when the activity state of groundwater is wet to water-seepage; and the water yield of every 10 meters of the tunnel section is within a range of 5 m³/h to 15 m³/h when the activity state of the groundwater is dripping. In a case of the two water discharging states, the possibility that part of rock mass structural plane slips under the lubricating effect of the water is increased, however, in general, the risk of TBM excavation affected by the groundwater is low. Wet and water-seepage tunnel section and the dripping tunnel section are small in water yield in the boring process, and the water can be completely drained through the conventional drainage equipment of the TBM. Some of the areas with high water seepage will have a certain influence on the application of the sprayed concrete, but this can be solved by simple treatment. Therefore, the grade of the surrounding rock is not reduced when the activity state of the groundwater is the wet to water-seepage state and the dripping state, but the observation should be strengthened when the rock mass intactness is relatively broken or broken.

The water yield of every 10 meters of the tunnel section is within the range of 15-50 m³/h when the activity state of the groundwater is linear water flow or rain-like water flow, the working environment in the tunnel begins to deteriorate, the risk that the structural plane of the rock is infiltrated by water and slips is increased, and TBM construction is affected. When the linear water flow or rain-like water flow occurs, the working staff are required to wear raincoats, and welding, drilling and spraying and mixing operations are affected, resulting in reduced operational efficiency and increased operational risk. When the rock mass intactness index is greater than 0.55, the rock mass stability is basically not affected by the linear water flow or rain-like water flow. When the rock mass intactness index is 0.45 to 0.55, under the influence of the linear water flow or rain-like water flow, steel arches and reinforcing meshes need to be additionally arranged for supporting on the basis of the original support design for the tunnel section with the high vault breaking degree. For a tunnel section with the rock mass intactness index of 0.35 to 0.45, under the influence of the linear water flow or the rain-like water flow, the distance between reinforcing meshes needs to be reduced, and longitudinal connection of steel arches and reinforcing meshes need to be properly increased. When the rock mass intactness index is below 0.35, the vault settlement is obviously increased under the influence of the linear water flow or rain-like water flow, and the steel arches needs to be densified in order to prevent the rock mass from instability and ensure the clearance of the tunnel. In conclusion, when the activity state of the groundwater is linear water flow or rain-like water flow, the influence of the groundwater on the surrounding rock with the rock mass intactness index of 0.35 and above is limited, construction adaptability degradation is avoided. However, when the rock mass intactness index is below 0.35, the construction adaptability rating should be reduced by one grade.

The water yield of every 10 meters of the tunnel section is within the range of 50 m³/h to 100 m³/h when the activity state of the groundwater is strand-like water flow, in this case, the working environment in the tunnel is further deteriorate, there is a high risk of water gushing in the tunnel, and the TBM construction is greatly affected. The water level near the mainframe is high due to large water yield, and the steel arch assembling, the track laying and the slag removing at the arch bottom are affected. At VII Package at KS Section of EH Project, upstream and downstream are respectively in reverse slope boring and forward slope boring, the water level near the upstream mainframe is low, and the water level near the downstream mainframe is high. Due to the influence of the high water level, the track laying precision at the downstream is poor, the track falling frequency of a rail transport train at the downstream is higher than that at the upstream, resulting in higher material delay time at the downstream than that at the upstream. After 24 hours of excavation, if the water volume has not decreased, geological drilling should be carried out and whether advanced grouting is needed should be decided according to the detection results. A polymer chemical grouting method was adopted for carrying out advanced water plugging at the pile numbers SD52+160.815 m to SD52+033.687 m at a certain section of SS of the EH Project, it took 36 days to cross a water-rich rock section of 139 meters, with a mean advance rate of about 3.86 m/d. By arranging water collectors and drainage pipes, the adverse effect of water on the sprayed concrete can be weakened, but when the rock mass intact index is smaller than 0.35, strand-like gushed water is usually accompanied by large-area rain-like water flow, and the weakening on the surrounding rock stability may refer to the previous activity state of the groundwater. In conclusion, when the activity state of the groundwater is strand-like water flow, the influence of the groundwater on the surrounding rock with the rock mass intactness index of 0.35 and above is deepened compared to the previous discharging state, but the construction adaptability degradation is avoided. When the rock mass intactness index is below 0.35, the construction adaptability rating should be reduced by one grade. If the geological examination result indicates that advanced grouting for water plugging is needed, the construction adaptability rating of the surrounding rock is T-V.

8. Influence of Hazard-Type Activity State of Groundwater on TBM Construction

When the activity state of groundwater is water gushing, the water yield every 10 meters in the tunnel section is over 100 m³/h. The influence form of gushed water is complex, and countermeasures to gushed water disasters in different projects are different. Therefore, it is necessary to summarize and discuss the relevant engineering cases where water gushing occurs.

9. Gaoligongshan Tunnel

(1) Water gushing event at parallel adit PDZK222+323

The influence range of the water gushing event was PDZK222+330 to 303, and the time for shutdown treatment was 14 days. The reason of shutdown was as follows: strand-like gushed water occurred at multiple positions of the right arch waist in a boring direction of the tunnel. Due to the infiltration of the water, the argillic alteration phenomenon of the tunnel face was serious, the TBM cutter head was difficult to rotate, and the cutter head torque exceeded the limit. The treatment solution was as follows: performing manual dredging to help the cutter head get rid of trouble, and reinforcing the surrounding rock by advanced grouting.

(2) Water Gushing Event at Parallel Adit PDZK222+271.814

The influence range of the water gushing event was PDZK222++277 to 246, and the time for shutdown treatment was 11 days. The reason of shutdown was as follows: the tunnel face and the tunnel arch portion discharged water, with the water yield of 150 m³/h, and due to high breaking degree and high weathering degree of the surrounding rock, the machine jamming risk was increased. The treatment solution was as follows: increasing pumping and drainage efforts, constructing advanced drainage holes, and furthermore, performing submerged chemical infusion and deep-hole advanced pipe shed simultaneously.

(3) Water Gushing Event at Parallel Adit PDZK221+917.957

The influence range of the water gushing event was PDZK221+920 to 901, and the time for shutdown treatment was 21 days. The reason of shutdown was as follows: the tunnel face discharged water, with the largest water yield of about 180 m³/h, and due to the high weathering degree of the surrounding rock, the tunnel face collapsed severely and the cutter head was difficult to start. The treatment solution was as follows: constructing the advanced pipe shed and performing grouting for water stop, and cleaning up the slag accumulation in the cutter head after the water volume is reduced.

(4) Water Gushing Event at Main Tunnel D1K224+234 to 180

The influence range of the water gushing event was D1K224+234 to 180, and the time for shutdown treatment was 41 days. The reason of shutdown was as follows: the buried depth of the tunnel in the influence range was relatively shallow, a catchment channel communicating with a surface pond was formed in the tunnel in construction, finally resulting in water gushing and surface collapse. The treatment solution was as follows: pumping and draining the surface pond to block the catchment channel, and performing advanced reinforcement via the parallel adit by using a circuitous pilot tunnel method.

The problem of weak surrounding rock in the Gaoligongshan Tunnel is prominent, and the influence of the weak surrounding rock is further aggravated by gushed water. Weathered rock and fault mud form slurry under the infiltration of water and finally wrap the cutter head to force the TBM to stop. In a subsurface buried section, due to the fact that construction was not conducted in the dry season, water gushing and surface collapse occurred. The shutdown caused by water gushing lasted for more than 10 days, and the shutdown may last for 41 days if a circuitous pilot tunnel is adopted.

10. TBM1 Boring Section of SS Tunnel of EH Project

When TBM1 bored to the mileage of 3+887, two water gushing points were generated at the fissure weak position of the left side tunnel wall in the boring direction, and the water was jet-like, and after being measured, the pressure of the gushed water was 0.9 MPa, and the water yield was 320 m³/h. The surrounding rock where the water gushing occurred was good in intactness and hard, and thus the stability was not affected greatly. However, due to the fact that the self-contained water pump of the TBM was limited in drainage capacity, water accumulation in the tunnel was serious, track laying operation was difficult to conduct, and cutter head was difficult to rotate.

The water volume was not reduced after the water gushing occurred in the tunnel for about a month. It was ascertained that the feed water source was from a reservoir near the tunnel. The water plugging mode had experienced “pressure relief hole+polyurethane material infusion”, “steel plate+geotextile plugging+polyurethane material infusion”, and “concrete pouring+cement paste backfilling”, but did not achieve the effect. Finally, the gushed water was successfully plugged by adopting “advanced drilling+cement+sodium silicate grouting”. A total of 58 days was spent from the occurrence of water gushing to successful plugging.

It can be seen from this case that the tunnel should be as far as possible from the area where the surface water source is developed at the design stage, and if difficult to avoid, large drainage redundancy should be considered in the TBM design. The choice of water plugging mode should be done in one step as much as possible to avoid repeated failure of water plugging and resulted severe delay in construction period.

11. TBM2 Boring Section of SS Tunnel of EH Project

During the trial boring of the project, water gushing occurred multiple times, with the maximum gushed water over 1,000 m³/h. In the subsequent boring process, water gushing was also encountered multiple times.

(1) Oct. 22, 2017 to Nov. 23, 2017

During such period, large water gushing occurred when the TBM bored to the mileage of SD52+160, with a maximum flow rate of 380 m³/h. A drilling and grouting method is adopted for water plugging, with the grouting materials including pure cement paste and cement+sodium silicate double-slurry. During water plugging, a total of 68 holes were drilled, with a cumulative length of 237.9 meters and an average hole depth of about 3.5 meters. As the TBM could not work normally during drilling, it stopped for 32 days, but the water plugging effect is good, with the plugging amount reaching about 70%.

(2) Nov. 24, 2017 to Dec. 30, 2017

Large water gushing accumulatively occurred seven times during such period, with a total flow rate of 690 to 830 m³/h, and a mean single flow rate of about 110 m³/h. Because the single flow rate was not large, polymer chemical grouting was adopted as a water plugging mode, which has a limited influence on the boring of the TBM so that TBM can bore continuously. The final water plugging effect achieved 60% of the plugging amount, boring of 139 meters was achieved in the water plugging period of 36 days, with a mean advance rate of 3.86 m/d.

It can be known from such case that the plugging mode for the water gushing has a great influence on the TBM construction. The continuous boring is hard to achieve during water plugging by using the drilling and grouting method, and the boring while plugging can be achieved by using the polymer chemical grouting method.

12. Case Summary

By summarizing the three cases above, the influence of water gushing on the TBM construction is determined by the water gushing amount, the surrounding rock state and water plugging measures.

When water gushing occurs in weakly broken surrounding rock, surrounding rock containing fault mud and surrounding rock with high weathering degree, safe boring of the TBM can be guaranteed only by performing cutter head dredging and fore poling except for timely pumping and draining. In this case, even if the flow rate of the gushed water is below 200 m³/h, the downtime may also exceed 10 days. If a circuitous pilot tunnel method is adopted for fore poling, the downtime may exceed 1 months.

When the surface water system is developed and the groundwater catchment channel is smooth, once water gushing occurs, the influence time may last for 1-2 months, and a good water plugging effect can be obtained by advanced grouting. In this case, even if the flow rate of gushed water is not large, the total gushing amount may exceed the drainage capacity of the TBM in a case that the catchment channel is not completely cut off. In the design, the tunnel should be as far as possible away from areas where the surface water system is developed, and planning should be made in advance during construction so as to underpass reservoirs, ponds and the like as much as possible in dry seasons.

The plugging mode for the gushed water has a great influence on the TBM construction. By adopting the drilling and grouting method, a waterproof curtain may be formed in front the tunnel face to enhance the stability of surrounding rock, but it is difficult to achieve continuous boring of the TBM during water plugging. As the drilling efficiency in the weak surrounding rock is higher than that in hard surrounding rock, the construction period using the drilling and grouting method changes greatly, about 10-60 days. By using the polymer chemical grouting method, boring while plugging can be achieved. From the application in the TBM2 Boring Section of SS Tunnel in EH Project, after using the polymer chemical grouting method, the advance rate of 3.86 m/d can be achieved while the gushed water with a flow rate of about 110 m³/h is effectively plugged.

Water gushing has a great influence on the TBM construction, once the water gushing occurs, the construction adaptability rating should be reduced to T-V.

13. Surrounding Rock Grade Correction Under Action of Groundwater

In conclusion, the TBM construction adaptability changes in different activity states of groundwater are summarized, and the surrounding rock grade correction method is finally obtained. However, the lithology, weathering degree and joint filler of the surrounding rock are also the decisive factors of the TBM affected by groundwater. For example, when the TBM bores in rocks such as mudstone with greatly reduced strength after encountering water, the supporting possibly needs to be reinforced after encountering groundwater; and when boring is carried out in a strong weathered zone, an altered zone and a broken zone with more fault mud, mud gushing disasters may occur due to the influence of groundwater. Due to the large randomness and contingency of such problems, its specific influence on the TBM construction should be determined based on engineering practice conditions.

Through the groundwater correction, by analyzing the degree of influence of different types and different magnitudes of water gushing on the TBM advance rate, a groundwater surrounding rock corrected classification table as shown in FIG. 5 is obtained for the tunnel with groundwater on the basis of the surrounding rock classification table.

Based on the project instances, the influences of the rock burst and groundwater on the TBM construction are summarized, and corresponding surrounding rock grade correction methods are provided accordingly. Upon discussion and analysis, the following conclusions are mainly drawn:

(1) The influence of weak rock burst on open type TBM construction is small, the construction adaptability rating of T-I, T-II and TI-III surrounding rocks is reduced by one grade after being affected by the weak rock burst, and the T-IV surrounding rock is not degraded. Under the influence of moderate rock burst, the construction adaptability rating of the T-I surrounding rock is reduced by two grades, the construction adaptability rating of the T-II surrounding rock is reduced by two grades, the construction adaptability rating of the T-III surrounding rock is reduced by one grade, and the construction adaptability rating of the T-IV surrounding rock is reduced by one grade. Intense rock burst and extremely intense rock burst have a great influence on the advance rate and safety of the open type TBM construction, and once the intense rock burst and extremely intense rock burst occur, the construction adaptability of the surrounding rock is directly rated to T-V.

(2) TBM construction adaptability of the surrounding rock in a dry state is not affected. The construction adaptability rating of the surrounding rock in the wet to water-seepage state and the dripping state is not degraded, but observation needs to be enhanced for the relatively broken to broken surrounding rock. The construction adaptability of the surrounding rock in the linear water flow state or rain-like water flow state is affected, the construction adaptability rating of the surrounding rock with the rock mass intactness index smaller than 0.35 is reduced by one grade, and observation needs to be enhanced. When the rock mass intactness index is smaller than 0.35, the construction adaptability rating of the surrounding rock in the strand-like water flow state is reduced by one grade, and if advanced water plugging is needed, the construction adaptability is reduced to grade T-V. The construction adaptability of the surrounding rock in a water gushing state is greatly affected, and once water gushing occurs, the construction adaptability is reduced to grade T-V.

Finally, it should be noted that the above is only a preferred embodiment of the present disclosure and is not intended to limit the present disclosure. Although the present disclosure has been described in detail with reference to the foregoing embodiments, it will be apparent to those skilled in the art that modifications can be made to the technical solutions described in the foregoing embodiments, and that equivalent replacement can be made to part of the technical features. Any modifications, equivalents, improvements and the like made within the spirit and principles of the present disclosure are intended to be included within the scope of the claims of the present disclosure.

1. A tunnel boring machine (TBM) TBM adaptive surrounding rock classification method oriented to advance rate and safety, the method comprising:
establishing a TBM adaptive surrounding rock classification table oriented to the advance rate and the safety, the TBM adaptive surrounding rock classification table at least describing correspondence relationships of grades of different surrounding rocks to respective TBM penetrations and respective supporting modes;
substituting rock uniaxial compressive strength and rock mass intactness index of a surrounding rock into the surrounding rock classification table to obtain a basic grade of the surrounding rock;
determining whether the basic grade of the surrounding rock needs to be corrected or not, and when correction is not needed, setting the basic grade of the surrounding rock to be a final grade of the surrounding rock;
when the correction is needed, performing rock burst correction or groundwater correction on the basic grade of the surrounding rock;
determining, in surrounding rock evaluation, which one of a corrected grade after the rock burst correction and a corrected grade after groundwater correction is poorer than another one
setting the poorer corrected grade determined in the surrounding rock evaluation as the final grade of the surrounding rock; and
searching the TBM adaptive surrounding rock classification table based on the final grade of the surrounding rock to obtain a supporting mode and a penetration of a TBM for penetrating the surrounding rock.

2. The TBM adaptive surrounding rock classification method oriented to advance rate and safety according to claim 1, wherein the advance rate in the surrounding rock classification table is obtained by collecting boring data of real engineering cases on site and analyzing advance rate AR under different surrounding rock conditions through using a big data statistical analysis method, and the advance rate is divided into five classes: AR-I, AR-II, AR-III, AR-IV and AR-V, with division criteria as follows: the advance rate of greater than 20 m/d is classified as class AR-I, the advance rate of greater than 15 and smaller than or equal to 20 m/d is classified as class AR-II, the advance rate of greater than 10 and smaller than or equal to 15 m/d is classified as class AR-III, the advance rate of greater than 5 and smaller than or equal to 10 m/d is classified as class AR-IV, and the advance rate of less than or equal to 5 m/d is classified as class AR-V.

3. The TBM adaptive surrounding rock classification method oriented to advance rate and safety according to claim 2, wherein TBM advance rate AR classes corresponding to various combinations of the uniaxial compressive strength UCS and the intactness index Kv of the surrounding rock are obtained by means of big data statistics and mathematical regression analysis, and the basic grade of the surrounding rock is obtained according to the compressive strength and the intactness index of the surrounding rock.

4. The TBM adaptive surrounding rock classification method oriented to advance rate and safety according to claim 3, wherein the basic grade of the surrounding rock is divided into five grades which are excellent T-I, good T-II, medium T-III, poor T-IV, and extremely poor T-V.

5. The TBM adaptive surrounding rock classification method oriented to advance rate and safety according to claim 1, wherein through the rock burst correction, degree of influence of different grades of rock burst on the advance rate and the construction safety of the TBM is obtained by analyzing a relationship between rock burst grade of a rock burst tunnel section and the advance rate and the construction safety, and a value by which the grade of the surrounding rock of the rock burst tunnel section is reduced is determined on a basis of the surrounding rock classification table, to obtain a rock burst surrounding rock classification correction table.

6. The TBM adaptive surrounding rock classification method oriented to advance rate and safety according to claim 1, wherein through the groundwater correction, by analyzing degree of influence of different types and different magnitudes of water inrush on the TBM advance rate and the construction safety, a groundwater surrounding rock corrected classification table is obtained for the tunnel with groundwater on a basis of the surrounding rock classification table.
